(19) United States
(12) Patent Application Publication
(10) Pub. No.: US 2008/0051580 A1
(43) Pub. Date: Feb. 28, 2008

(54) Process to Prepare Camptothecin Derivatives and Novel Intermediate and Compounds Thereof

(76) Inventor: Ragina Naidu, Burnaby (CA)

Correspondence Address: KLARQUIST SPARKMAN, LLP, 121 SW SALMON STREET, SUITE 1600, PORTLAND, OR, 97204, US

(21) Appl. No.: 11/628,389

(22) PCT Filed: Jun. 3, 2005
(86) PCT No.: PCT/US05/19700

§ 371 Date: Sep. 14, 2007

Related U.S. Application Data
(63) Continuation-in-part of application No. 10/861,097, filed on Jun. 4, 2004, now abandoned.

(30) Foreign Application Priority Data
Jun. 9, 2004 (SE) 0401460-1

(51) Int. Cl. C07D 405/14

(52) U.S. Cl. 546/48

(57) ABSTRACT

New processes are disclosed for the preparation of camptothecin derivatives, such as, irinotecan and topotecan, as well as new intermediates and compositions thereof.

### BACKGROUND OF THE INVENTION

1. Field of the Invention

The present invention relates to new processes to prepare camptothecin derivatives, such as, irinotecan and topotecan, and novel intermediate and compounds related thereof.

2. Description of the Related Art

Camptothecin 1 is a pentacyclic alkaloid that was isolated by Wall et al. in the early 1960s from the Chinese tree, Camptotheca acuminate (Nyssaceae). The compound raised immediate interest as a potential cancer chemotherapeutic agent due to its impressive activity against a variety of tumors. However, a shortcoming of camptothecin as an anti-cancer agent was its poor solubility in water. To overcome the solubility problem, the sodium salt was synthesized by hydrolysis of the lactone ring. This sodium salt forms an equilibrium with the ring-closed lactone form. As its sodium salt, camptothecin was moved to clinical trials and promising activity was initially observed. However severe side effects and drug-related toxicities finally led to discontinuation of the clinical program.

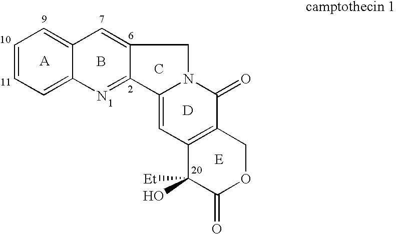

Stimulated by the challenging structure and its very interesting biological activity, synthetic approaches to camptothecin were developed. During semi-synthetic and total-synthetic chemistry programs, the particular importance of the lactone ring and the C20 (S)-configuration for good biological activity was recognized. In contrast, modifications in the A-ring and B-ring, particularly in the C9, C10 and C11 positions, were tolerated and led to improved analogues.

Second-generation camptothecin derivatives have been optimized for improved water solubility to facilitate intravenous drug administration. Highlights resulting from various programs at different companies and institutions are irinotecan 2 and topotecan 3, two compounds which are successfully used in clinical practice, and SN-38 4, exatecan 5, liposomal lurtotecan 6 (OSI-211) and CKD-602 7, which are in advanced stages of clinical development. The chemical structures of these compounds are shown in FIGS. 1A and 1B.

Irinotecan 2 was discovered at Yakult Honsha and was first approved in Japan in 1994 (Camptotesin®) for lung, cervical and ovarian cancer. Today it is marketed in the U.S. by Pharmacia (Camptosar®) and by Aventis in Europe (Campto®). Irinotecan 2 is a prodrug which is cleaved in vivo by carboxylic esterases, particularly by hCE-2, to release the active metabolite SN-38 4. An intermediate for some of above compounds is 10-hydroxy camptothecin, which may be prepared as set forth in the U.S. Pat. No. 4,473,692. According to this patent, 10-hydroxy camptothecin may alternatively be prepared by subjecting a N1-oxide intermediate of camptothecin to UV irradiation.

Because of the promising biological activity shown by this class of compounds, and also because of the successful medical applications for this class of compounds, there is a need in the art for new and improved synthetic methods to make compounds within this class. The present invention addresses this need and provides further related advantages as set forth herein.

### BRIEF SUMMARY OF THE INVENTION

In brief, the present invention is related to improved processes to prepare camptothecin derivatives such as irinotecan and topotecan, and new intermediates and related compounds thereof.

In one embodiment, a method to introduce a hydroxyl group on the C10 position of the A ring of a camptothecin derivative is provided, comprising exposing a compound of formula I to oxidative conditions to provide a compound of formula II: 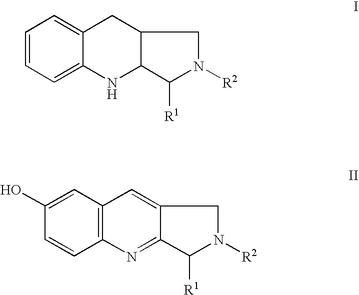
wherein R¹ and R² are the same or different and the same or different and are independently hydrogen, hydroxyl or an organic group.

In a specific embodiment of the foregoing, 1,2,6,7-tetrahydrocamptothecin is converted to 10-hydroxy camptothecin in a one-step oxidation.

In a further embodiment, a method of silylating the N1 position of a camptothecin derivative is provided, comprising subjecting a compound of formula IIa to silylation conditions to thereby provide a compound of formula IIIa, 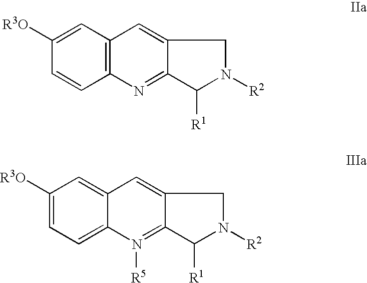
wherein R¹ and R² are the same or different and are independently hydrogen, hydroxyl or an organic group, R³ is hydrogen or a hydroxyl protecting group, R⁵ is a silyl group, and wherein the compound of formula IIIa is associated with a counterion.

In a specific embodiment of the foregoing, the silylating reagent is t-butyldimethylsilyl triflate.

In yet another embodiment, a method to alkylating the C7 position of the B ring of a camptothecin derivative is provides, comprising exposing a compound of formula IIIa to alkylation conditions, followed by oxidation conditions, to provide a compound of formula IV 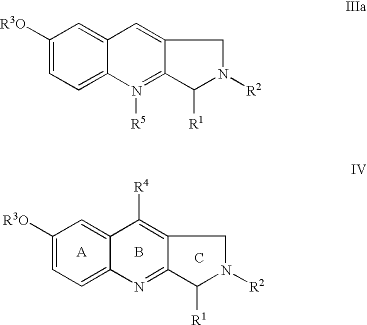
wherein, R¹ and R² are the same or different and are independently hydrogen, hydroxyl or an organic group, R³ is hydrogen or a hydroxyl protecting group, R⁴ is an alkyl group, R⁵ is a silyl group, and wherein the compound of formula IIIa is associated with a counterion.

In a specific embodiment of the foregoing, 7-ethyl-10-hydroxy camptothecin is prepared, which is further converted to irinotecan.

In another further embodiment, the present invention provides a method comprising exposing a compound of formula III to silylating conditions to provide a compound of formula V 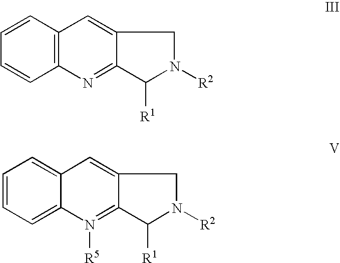
wherein, R¹ and R² are the same or different and are independently hydrogen, hydroxyl or an organic group, R⁵ is a silyl group, and wherein the compound of formula V is associated with a counterion.

In a specific embodiment of the foregoing, N-silyl camptothecin is provided according to the method disclosed.

In yet another further embodiment, the present invention provides a method comprising exposing a compound of formula V to oxidation conditions, to afford a compound of formula II 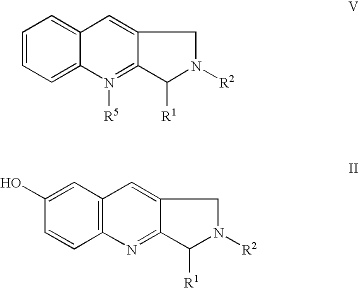
wherein, R¹ and R² are the same or different and are independently hydrogen, hydroxyl or an organic group, R⁵ is a silyl group, and wherein the compound of formula V is associated with a counterion.

In a specific embodiment of the foregoing, N-silyl camptothecin is converted to 10-hydroxy camptothecin under the oxidation condition.

In yet another further embodiment, the present invention provides method comprising exposing a compound of formula II to formylation conditions, to afford a compound of formula VII 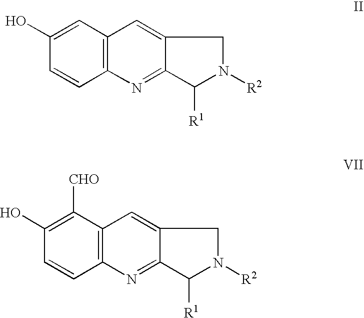
wherein R¹ and R² are the same or different and are independently hydrogen, hydroxyl or an organic group.

In yet another further embodiment, the present invention provides a method comprising exposing a compound of formula VII to reductive amination conditions, to afford a compound of formula VIII 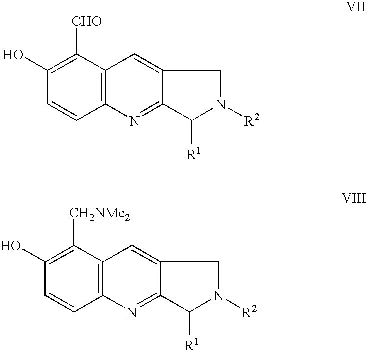
wherein R¹ and R² are the same or different and are independently hydrogen, hydroxyl or an organic group.

In yet another further embodiment, the present invention provides a compound of the formula, or a stereoisomer, or a salt thereof, 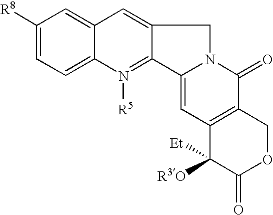
wherein, R⁸ is hydrogen or OR³, R³ and R^{3′} are the same or different and are independently a hydroxyl protecting group, R⁵ is a silyl group.

In a specific embodiment of the foregoing, R⁸ is hydrogen.

In yet another further embodiment, the present invention provides a process comprising oxidizing a starting material selected from camptothecin and a derivative thereof, in the presence of an organic peroxide, to form the corresponding 1-oxide compound.

In yet another further embodiment, the present invention provides a process comprising exposing camptothecin-1-oxide or a derivative thereof, to oxidation conditions, to introduce a hydroxyl group to the C10 position of the corresponding camptothecin or the derivative thereof while remove the oxide group, the oxidation conditions comprising an oxidizing reagent in the absence of directed irradiation with UV light.

In yet another further embodiment, the present invention provides a process comprising exposing a starting material selected from the group consisting of camptothecin, 10-hydroxy camptothecin, camptothecin-1-oxide, and a derivative thereof, to alkylation conditions to form a corresponding 7-alkyl compound.

In yet another further embodiment, the present invention provides a process for preparing 7-ethyl-10-hydroxy camptothecin or a stereoisomer or a salt thereof using 10-hydroxy camptothecin as a starting material comprising: exposing 10-hydroxy camptothecin to a silylation condition to provide a 10-hydroxy-N-silyl camptothecin intermediate, followed by reacting the 10-hydroxy-N-silylated intermediate with ethyl magnesium halide in the presence of an ether solvent to provide a 7-ethyl-10-hydroxy-N-silyl camptothecin intermediate, which is then subjected to an oxidation condition to remove the silyl group to provide 7-ethyl-10-hydroxy camptothecin.

In yet another further embodiment, the present invention provides a process of preparing irinotecan comprising: catalytically hydrogenating camptothecin to provide 1,2,6,7-tetrahydrocamptothecin, followed by oxidizing 1,2,6,7-tetrahydrocamptothecin to provide 10-hydroxy camptothecin, which is then treated with a silylating reagent to introduce a silyl group to the N1 position, the resulting 10-hydroxy-N-silyl camptothecin is then reacted with a ethylmagnesium halide to provide 7-ethyl-10-hydroxy-N-silylcamptothecin, which is further oxidized to remove the silyl group, followed by reacting the resulting 7-ethyl-10-hydroxy-N-silyl camptothecin with piperidinopiperidinecarbamyl chloride to provide irinotecan.

In yet another further embodiment, the present invention provides an alternative process of preparing irinotecan comprising: protecting camptothecin with a hydroxyl protecting group on the C20 position; reacting the protected camptothecin with a silylating reagent to introduce a silyl group to the N1 position thereby provide N-silylcamptothecin with the C20 hydroxyl protected, which is then reacted with ethylmagnesium halide to provide a C20 protected 7-ethyl-N-silylcamptothecin, the C20 protected 7-ethyl-N-silylcamptothecin is then oxidized to remove the silyl group from the N1 position and to introduce a hydroxyl group on the C10 position, the C20 position is then deprotected to provide 7-ethyl-10-hydroxy camptothecin, which is reacted with piperidinopiperidinecarbamyl chloride to provide irinotecan.

In yet another further embodiment, the present invention provides a process of preparing topotecan comprising: reacting camptothecin with a silylating reagent to introduce a silyl group to the N1 position thereby to provide N-silylcamptothecin, followed by oxidizing the resulting N-silylcamptothecin to provide 10-hydroxy camptothecin, followed by treating 10-hydroxy camptothecin in a formylation condition to provide 9-formyl-10-hydroxy camptothecin, which is then subjected to a reductive amination condition to provide topotecan.

These and other aspects of the invention will be apparent upon reference to the attached and following detailed description.

### BRIEF DESCRIPTION OF THE DRAWINGS

FIGS. 1A and 1B show the chemical structures of camptothecin 1, and various derivatives of camptothecin, specifically irinotecan 2, topotecan 3, SN-38 4, exatecan 5, lurtotecan 6, and CKD-602 7.

FIGS. 2A and 2B illustrate a chemical synthesis of irinotecan 2 from camptothecin 1 according to the present invention, where the present invention provides, as separate aspects of the invention, for the conversion of compound 1 to compound 8, for the conversion of compound 8 to compound 9, for the conversion of compound 9 to either or both of compound 10 and compound 11, for the conversion of compound 10 to compound 4, for the conversion of compound 11 to compound 4, for the conversion of a mixture of compounds 10 and 11 to compound 4, and for the conversion of compound 4 to irinotecan 2.

FIG. 3 illustrates a chemical synthesis of irinotecan 2 from camptothecin 1 according to the present invention, where the present invention provides, as separate aspects of the invention, for the conversion of 1 to either compound 13 or compound 14, where compound 14 may also be prepared from compound 13 , and the conversion of compound 14 to compound 15 , and the conversion of compound 15 to irinotecan 2.

FIG. 4 illustrates a chemical synthesis of topotecan 3 from camptothecin 1 according to the present invention, where the present invention provides, in separate aspects, for the conversion of compound 1 to topotecan 3 via a novel intermediate 14.

FIG. 5 illustrates chemical syntheses of derivatives of camptothecin, which employ camptothecin-1-oxide as an intermediate.

FIG. 6 illustrates a chemical synthesis of SN-38 4 from camptothecin 1.

### DETAILED DESCRIPTION OF THE INVENTION

The present invention provides synthetic methods and compounds produced by, or using, such synthetic methods. The compounds are useful as intermediates in the preparation of derivatives of camptothecin, where the intermediates may also have desirable biological activity.

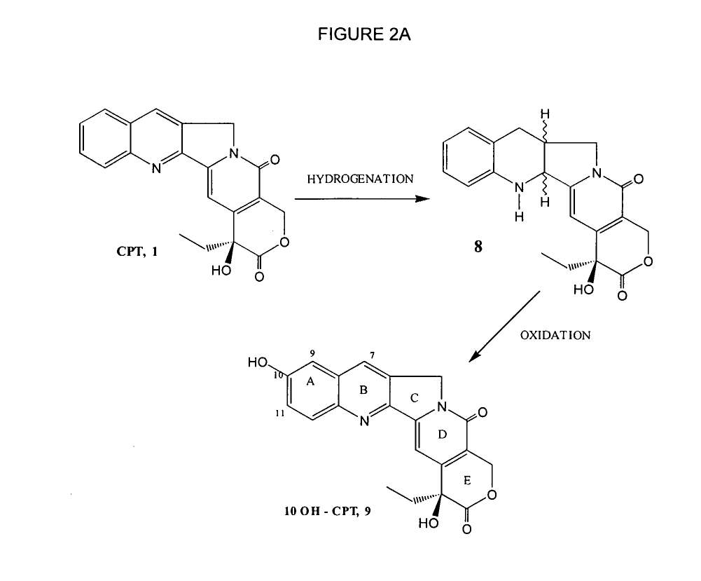

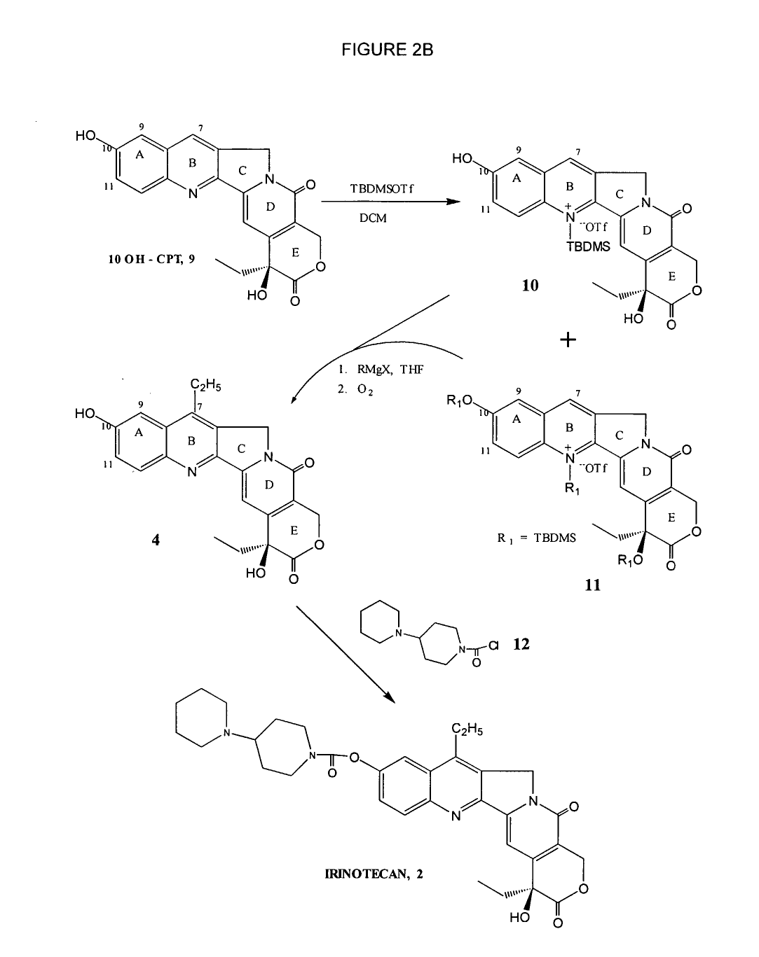

A series of synthetic methods according to the present invention is shown in FIGS. 2A and 2B. In one aspect, the present invention provides a novel route for the preparation of 10-hydroxy camptothecin via a hydrogenation product of camptothecin. Camptothecin itself is a well known chemical available from many sources. For example, it may be isolated from plant material as described by Wall et al. (JACS 88:3888, 1966). Alternatively, it may be synthesized from commercially available materials, see, e.g., Corey et al., JACS 40:2140, 1975; Bradley et al. JOC 41:699, 1976; Walraven et al., Tetrahedron 36:321, 1980.

Camptothecin→10-Hydroxy Camptothecin

According to one aspect of the present invention, 10-hydroxy camptothecin is prepared by the oxidation of a hydrogenation product of camptothecin (compound 8 as shown below). 8 may be subjected to oxidation conditions to achieve, in one step, both the re-aromatization of the B ring and introduction of a hydroxyl group onto the C10 position on the A ring.

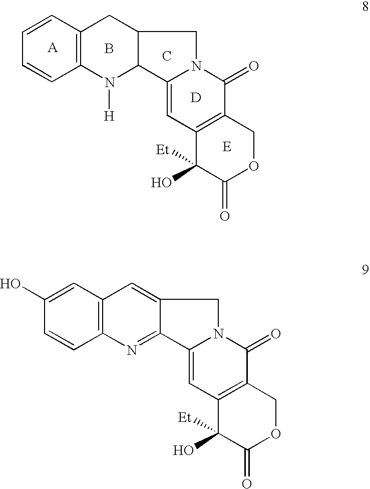

In particular, the hydrogenation step can be carried out under the atmospheric pressure in the presence of a suitable catalyst, such as palladium hydroxide or palladium oxide. Suitable solvent includes glacial acetic acid.

10-hydroxycamptothecin 9 is then conveniently converted from 8 under an oxidation condition. The oxidizing reagents may be palladium diacetate or lead (IV) acetate in the presence of a protic acid such as acetic acid or trifluoroacetic acid; or Jones reagent; or pyridinium chlorochromate. As used herein, protic acid refers to an acid that yields an H⁺ ion. Compared to the process disclosed in the U.S. Pat. No. 4,473,692, in which fuming nitric acid is used to functionalize the C10 position prior to several steps of conversions in order to afford 9, the above process can be advantageously carried out under a relatively mild condition and in a shortened synthetic pathway.

The oxidation reaction illustrated by the conversion of 8 to 9 is independent of the stereochemical arrangement of the substituents on the E ring. Moreover, the oxidation reaction is also independent of the presence of the D and E rings. In other words, the D and/or E rings need not have been formed at the time that the B ring is aromatized and the A ring becomes hydroxyl-substituted according to the present invention.

Thus, generally speaking, the present invention provides a method comprising subjecting a compound of formula I to oxidative conditions to provide a compound of formula II 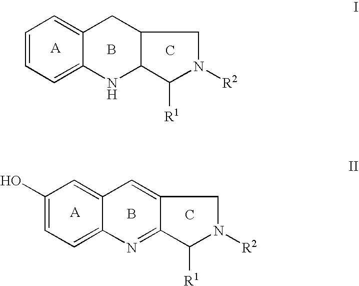
wherein R¹ and R² are the same or different and independently hydrogen, hydroxyl or an organic group.

An “Organic group” as used herein is broadly defined as any stable carbon-based group comprising one or more of elements selected from hydrogen, nitrogen, oxygen, sulfur, phosphorous, and halogen in their appropriate valencies. Generally speaking, the types of the organic groups in the generic structures I and II will not affect the hydrogenation and oxidation process, because the reactions are selective with respect to the saturation/re-aromatization of the B ring and hydroxylation of the C10 position of the A ring. Furthermore, at least in some incidences, a functionality in the organic groups that is susceptible to the hydrogenation process is likely to be restored after the oxidation step. In any event, it will be within the knowledge of one skilled in the art, to provide the necessary protection to a particular functionality that may undergo undesirable reduction or oxidation. Suitable protecting groups may be identified by consulting treatises such as “Protecting Groups in Organic Synthesis” (J. R. Hanson, Blackwell Science, Inc. 2000), or Protective Groups in Organic Synthesis” (P. G. Wuts & T. Greene, John Wiley & Son Inc. 1999).

More specifically, R¹ and R² are the same and different and independently alkyl, alkenyl, alkynyl, alkoxy, acyl, formyl, aryl, heteroaryl or heterocycle. As used herein, these terms have the following meanings:

“Alkyl” refers to an optionally substituted hydrocarbon structure having from 1 to 14 carbon atoms, wherein the carbons are arranged in a linear, branched, or cyclic manner, including combinations thereof. Lower alkyl refers to alkyl groups of from 1 to 6 carbon atoms. Examples of lower alkyl groups include methyl, ethyl, propyl, isopropyl, butyl, s- and t-butyl and the like. “Cycloalkyl” is a subset of alkyl and includes cyclic hydrocarbon groups of from 3 to 14 carbon atoms. Examples of cycloalkyl groups include cyclopropyl, cyclobutyl, cyclopentyl, norbornyl, adamantyl and the like. When an alkyl residue having a specific number of carbons is named, all geometric isomers having that number of carbons are intended to be encompassed; thus, for example, “butyl” is meant to include n-butyl, sec-butyl, isobutyl and t-butyl; propyl includes n-propyl and isopropyl.

“Alkenyl” refers to an alkyl group having at least one site of unsaturation, i.e., at least one double bond.

“Alkynyl” refers to an alkyl group having at least one triple bond between adjacent carbon atoms.

“Alkoxy” and “alkoxyl” both refer to moieties of the formula —O-alkyl. Examples include methoxy, ethoxy, propoxy, isopropoxy, cyclopropyloxy, cyclohexyloxy and the like. Lower-alkoxy refers to groups containing one to six carbons. The analogous term “aryloxy” refers to moieties of the formula —O-aryl.

“Acyl” refers to moieties of the formula —C(═O)-alkyl. One or more carbons in the acyl residue may be replaced by nitrogen, oxygen or sulfur as long as the point of attachment to the parent remains at the carbonyl. Examples include acetyl, benzoyl, propionyl, isobutyryl, t-butoxycarbonyl, benzyloxycarbonyl and the like. Lower-acyl refers to groups containing one to six carbons.

“Aryl” refers to an optionally substituted aromatic carbocyclic moiety such as phenyl or naphthyl.

“Heteroaryl” refers to a 5- or 6-membered heteroaromatic ring containing 1-3 heteroatoms selected from O, N, or S; a bicyclic 9- or 10-membered heteroaromatic ring system containing 1-3 heteroatoms selected from O, N, or S; or a tricyclic 13- or 14-membered heteroaromatic ring system containing 1-3 heteroatoms selected from O, N, or S. The heteroaryl may be optionally substituted with 1-5 substituents. Exemplary aromatic heterocyclic rings include, e.g., imidazole, pyridine, indole, thiophene, benzopyranone, thiazole, furan, benzimidazole, quinoline, isoquinoline, quinoxaline, pyrimidine, pyrazine, tetrazole and pyrazole.

“Heterocycle” means a 5- to 7-membered monocyclic, or 7- to 10-membered bicyclic, heterocyclic ring which is either saturated, unsaturated, or aromatic, and which contains from 1 to 4 heteroatoms independently selected from nitrogen, oxygen and sulfur, and wherein the nitrogen and sulfur heteroatoms may be optionally oxidized, and the nitrogen heteroatom may be optionally quaternized, including bicyclic rings in which any of the above heterocycles are fused to a benzene ring. The heterocycle may be optionally substituted with 1-5 substituents. The heterocycle may be attached via any heteroatom or carbon atom. Heterocycles include heteroaryls as defined above. Thus, in addition to the heteroaryls listed above, heterocycles also include morpholinyl, pyrrolidinonyl, pyrrolidinyl, piperidinyl, piperazinyl, hydantoinyl, valerolactamyl, oxiranyl, oxetanyl, tetrahydrofuranyl, tetrahydropyranyl, tetrahydropyridinyl, tetrahydropyrimidinyl, tetrahydrothiophenyl, tetrahydrothiopyranyl, tetrahydropyrimidinyl, tetrahydrothiophenyl, tetrahydrothiopyranyl, and the like.

“Formyl” refers to the moiety —C(═O)H.

“Halogen” refers to fluoro, chloro, bromo or iodo.

The term “substituted” as used herein means any of the above groups (e.g., alkyl, alkoxy, acyl, aryl, heteroaryl and heterocycle) wherein at least one hydrogen atom is replaced with a substituent. In the case of an oxo substituent (“═O”) two hydrogen atoms are replaced. Substituents include halogen, hydroxy, oxo, alkyl, substituted alkyl, aryl, substituted aryl, heteroaryl, substituted heteroaryl, heterocycle, substituted heterocycle, —NRₐR_{b}, —NRₐC(═O)R_{b}, —NR_{c}C(═O)NRₐR_{b}, —NRₐC(═O)OR_{b}, —NRₐSO₂R_{b}, —ORₐ, —C(═O)Rₐ, —C(═O)ORₐ, —C(═O)NRₐR_{b}, —OC(═O)Rₐ, —OC(═O)ORₐ, —OC(═O)NRₐR_{b}, —NRₐSO₂R_{b}, or a radical of the formula —Y—Z—Rₐ where Y is alkanediyl, substituted alkanediyl or a direct bond, alkanediyl refers to a divalent alkyl with two hydrogen atoms taken from the same or different carbon atoms, Z is —O—, —S—, —S(═O)—, —S(═O)₂—, —N(R_{b})—, —C(═O)—, —C(═O)O—, —OC(═O)—, —N(R_{b})C(═O)—, —C(═O)N(R_{b})— or a direct bond, wherein Rₐ, R_{b} and R_{c} are the same or different and independently hydrogen, amino, alkyl, substituted alkyl (including halogenated alkyl), aryl, substituted aryl, heteroaryl, substituted heteroaryl, heterocycle, or substituted heterocycle, or wherein Rₐ and R_{b} taken together with the nitrogen atom to which they are attached form a heterocycle or substituted heterocycle.

Alternatively, R¹ and R² groups may together with the atoms to which they are attached form a heterocycle. Specific examples include R¹ and R² groups forming the D and E rings of camptothecin or a derivative thereof. Alternatively, R¹ and R² groups may together represent a modified D and/or modified E ring of camptothecin or a derivative thereof, e.g., an E ring having a protected hydroxyl group in lieu of the naturally-occurring free hydroxyl group. As another alternative, R¹ and R² together may represent an intact D ring but an open-chain E ring, i.e., a pre-E ring structure wherein the lactone has not yet formed. Generally, and in one aspect of the invention, R¹ and R² are precursor groups used in the preparation of the D and E rings of camptothecin or a derivative thereof such as irinotecan or topotecan.

N-Silyl Camptothecin Compounds and Reactions Thereof

In another aspect, the present invention provides camptothecin compounds, and derivatives thereof, wherein the nitrogen at the 1 position is substituted with a silyl group. Such compounds are very useful as intermediates in the preparation of 7-alkylated camptothecin and derivatives of 7-alkylated camptothecin, as illustrated in FIG. 2B.

Thus, one aspect of the invention provides a method comprising subjecting a compound of formula IIa to silylation conditions to thereby provide a compound of formula IIIa, 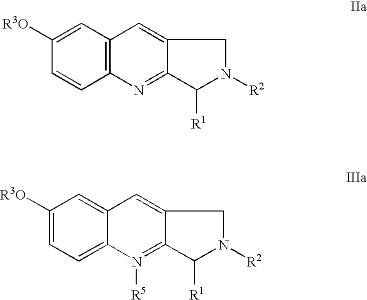
wherein R¹ and R² are the same or different and are independently hydrogen, hydroxyl or an organic group as defined above, R³ is hydrogen or a hydroxyl protecting group, and R⁵ represents a silyl group, wherein the compound of formula IIIa is associated with a counterion. Counterion as used herein refers to any chemically compatible species used for charge balance. In one embodiment the counterion is triflate. In another embodiment, the counterion is halide (including chloride, bromide and iodide). Hydroxyl protecting group as used herein refers to any derivative of a hydroxyl group known in the art which can be used to mask the hydroxyl group during a chemical transformation and later removed under conditions resulting in the hydroxyl group being recovered without other undesired effects on the remainder of the molecule containing the hydroxyl group. Many esters, acetals, ketals and silyl ethers are suitable protecting groups. Representative esters include acetyl, propionyl, pivaloyl and benzoyl esters. Representative ethers include allyl, benzyl, tetrahydropyranyl, ethoxyethyl, methoxymethyl, and benzyloxymethyl ethers. Representative acetals and ketals include acetonide, ketal groups derived from cyclic ketones such as cyclohexanone, from benzaldehyde or from p-O-methoxybenzaldehyde. Representative silyl ethers include trimethylsilyl, t-butyldimethylsilyl, and t-butyldiphenylsilyl ethers.

As with the oxidation reaction discussed above, this silylation reaction may be conducted on compounds of formula IIa wherein R¹ and R² together with the atoms to which they are attached represent a heterocycle. More specifically, R¹ and R² together with the atoms to which they are attached represent the D and E rings of camptothecin or a derivative thereof, such as compounds of the following formulae:

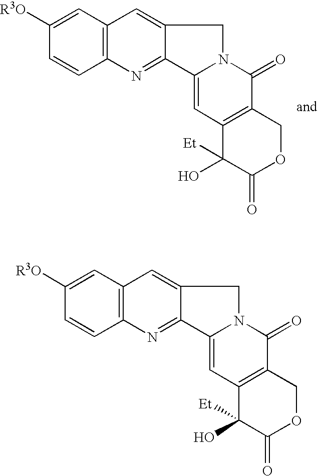

However, the silylation reaction may also be conducted on compounds wherein R¹ and R² represent precursor groups that will be used to generate the D and E rings of camptothecin or a derivative thereof.

The R³ group in the structures IIa and IIIa represents hydrogen or a hydroxyl protecting group. Thus, the compound of formula IIa may, optionally, have a protected hydroxyl group at the 10 position. Even if the hydroxyl group at the 10 position is unprotected, it may gain a protecting group during the silylation reaction in the event the hydroxyl group reacts with the silylating reagent. When the silylating reagent is t-butyl-dimethyl silyl triflate, some degree of silylation typically occurs at the 10 hydroxyl position in addition to the N1-silylation.

A preferred silylating reagent is t-butyl dimethylsilyl triflate. Other silanes having leaving groups may also be used in the practice of the present invention. The leaving group of a silylating reagent, such as triflate or halide may become the counterion following the silylation. Additional examples of suitable silylating reagents include trimethylsilyl chloride (TMSCl), trimethylsilyltriflate (TMSOTf), t-butyldiphenylsilyltriflate (TBDPSOTf), and triisopropylsilyl chloride (TIPSCl).

In another aspect, the present invention provides a method comprising exposing a compound of formula IIIa to alkylation conditions, followed by oxidation conditions, to provide a compound of formula IV 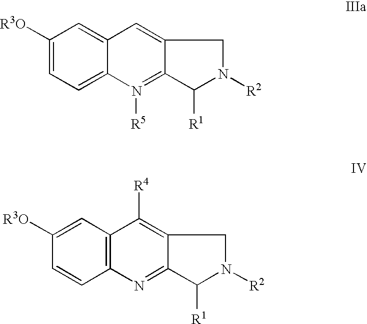
wherein R¹ and R² are the same or different and are independently hydrogen, hydroxyl or an organic group, R³ is hydrogen or a hydroxyl protecting group, R⁴ is an alkyl group, and R⁵ represents a silyl group, and wherein the compound of formula IIIa is associated with a counterion.

As with the oxidation and silylation reactions discussed above, the present alkylation reaction may be conducted on compounds of formula IIIa wherein R¹ and R² together with the atoms to which they are attached represent a heterocycle. More specifically, R¹ and R² together with the atoms to which they are attached represent the D and E rings of camptothecin or a derivative thereof, such as compounds of the following formulae:

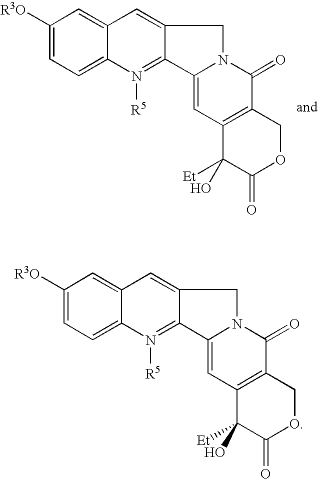

However, the silylation reaction may also be conducted on compounds wherein R¹ and R² represent precursor groups that will be used to generate the D and E rings of camptothecin or a derivative thereof.

Suitable alkylation conditions comprise the use of a Grignard reagent, i.e., an alkyl magnesium bromide compound or the equivalent. The alkyl group will typically have 2-8 carbons, although the method readily allows for the use of Grignard reagents having alkyl groups with more than 8 carbons. The Grignard reaction will typically be run in a suitable inert solvent, such as an ether, e.g., tetrahydrofuran. The alkylation reaction can be carried out at low temperature, which is in the range of room temperature to −78° C., preferably between −30 to −40° C.

Following the Grignard reaction, the resulting reaction mixture is treated with an oxidizing reagent, for example, oxygen, to remove the silyl group.

Thus, in a preferred embodiment, a compound of the formula 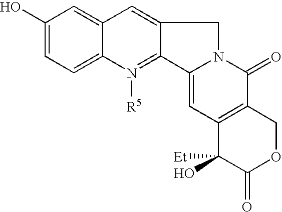
is exposed to ethylmagnesium chloride or ethylmagnesium bromide in THF, to thereby introduce an ethyl group to the 7 position. The resulting reaction mixture is then treated with oxygen to remove the R⁵ group and SN 38 4 is obtained: 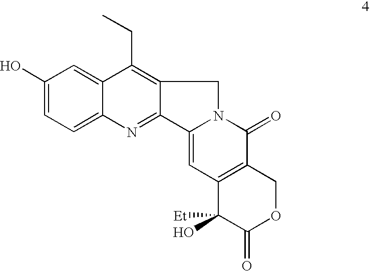
Preparation of Irinotecan 2 and Related Compounds

In another aspect, the present invention provides a one pot procedure for converting the 10-hydroxy group to a 10-urethane group. In this aspect of the invention, 7-alkyl-10-hydroxy camptothecin (e.g., 4), or a derivative thereof, prepared according to the processes as described above, is treated with a substituted phosgene compound 12 to provide the corresponding urethane compound. This reaction is illustrated in FIG. 2B by the conversion of 4 to irinotecan 2. Details of this reaction are described in U.S. patent application entitled “Process to Prepare Camptothecin Derivatives” by Ragina Naidu, filed on May 28, 2004 (attorney's docket number: 740082.413), which is incorporated herein by reference in its entirety.

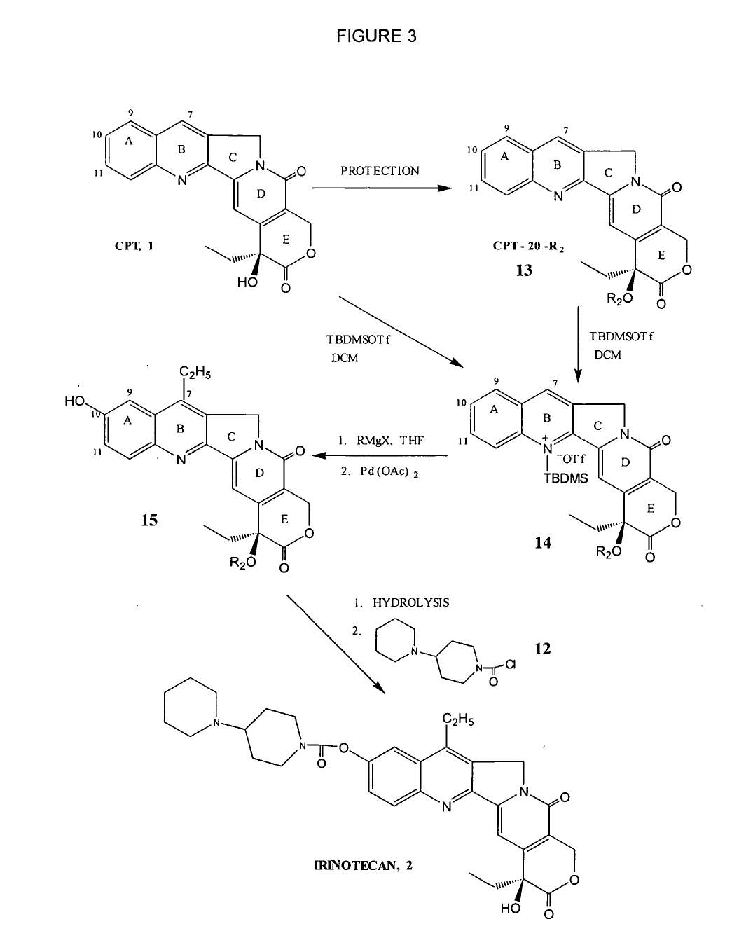

An alternative way of preparing irinotecan 2 according to the present invention is illustrated in FIG. 3. Here, a comprehensive synthetic pathway is shown for the preparation of irinotecan 2 via a novel N-silylated camptothecin intermediate 14.

In one aspect, the present invention provides an initial step of converting camptothecin 1 to the corresponding protected alcohol 13, wherein R⁷ represents a hydroxyl protecting group.

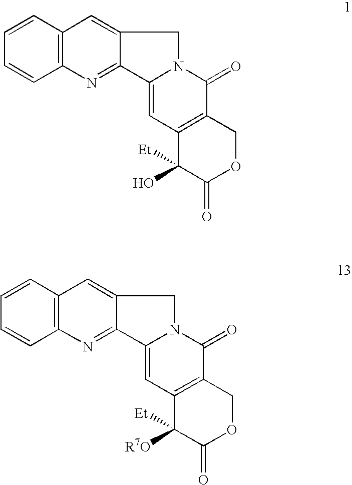

The protection reaction may be accomplished using standard protection conditions as outlined in, e.g., Protecting Groups in Organic Synthesis, supra. Following the protection step, 13 may be exposed to silylation conditions as set forth previously, to provide the N-silylated intermediate 14, wherein R⁵ represents a silyl group, e.g., t-butyl dimethyl silyl.

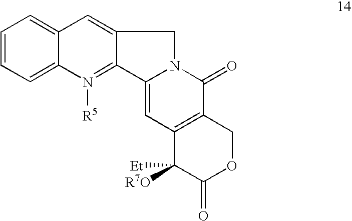

In another aspect, the present invention provides for a direct conversion of camptothecin to the intermediate 14 via a single silylating step, in which, the same silylating reagent provides both a silyl group at the N1 position and protection to the hydroxyl group located on the C20 position on the E ring. Accordingly, R⁵ and R⁷ are identical in compound 14 prepared by this method. Suitable silylating reagents are as set forth above.

Following the silylation, 14 may be converted to the corresponding 7-alkyl-10-hydroxy camptothecin 15 in a two-step process. An initial treatment with a Grignard reagent introduces an alkyl group at the C7 position. Subsequent reaction with palladium acetate or lead(IV) acetate introduces a hydroxyl group at the C10 position. 15 may be readily converted to irinotecan 2 following the same procedure as set forth earlier in connection with FIGS. 2A and 2B.

Preparation of Topotecan 3 and Related Compounds

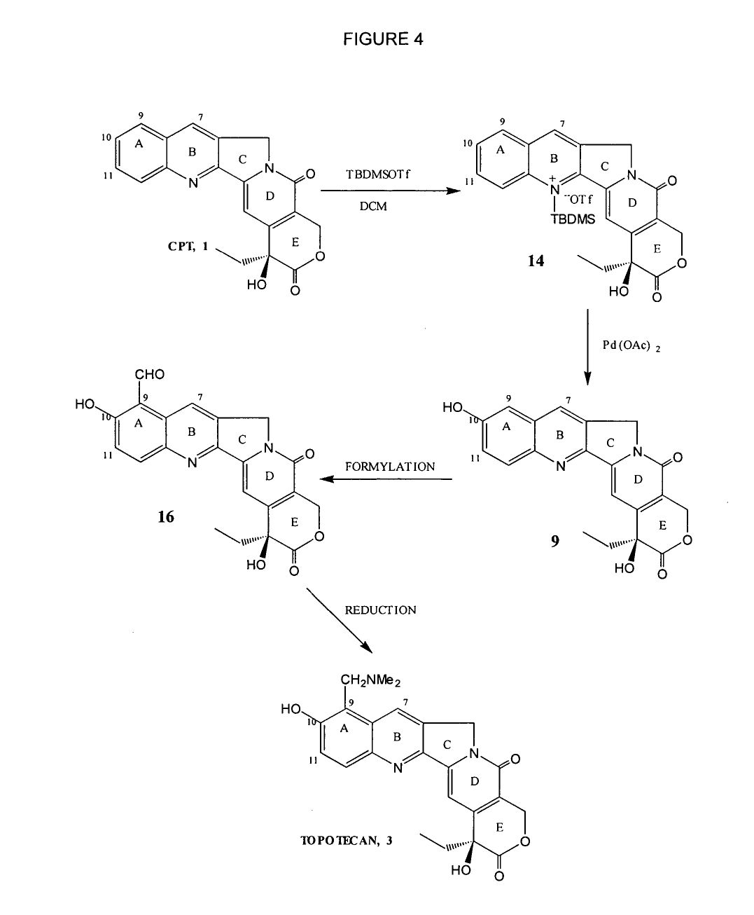

Another camptothecin derivative, topotecan 3 can be prepared via the novel intermediate 14 as illustrated in FIG. 4. In one aspect of the invention, 10-hydroxy camptothecin 9 is first obtained by treating 14 with an oxidizing reagent to introduce a hydroxyl group to the C10 position of camptothecin. The silyl group at the N1 position is also removed during this step. This route affords an alternative approach to the preparation of 9 compared to that shown in FIG. 2A.

In order to introduce the carbon substitution at the C9 position of camptothecin, a formylation reaction may be conducted to thereby place an aldehyde group at C9 to form 16. Exposure of 16 to reducing conditions affords topotecan 3 having a dimethylaminomethyl group at C9.

The oxidation, formylation and reduction reaction may be performed starting with camptothecin as shown in FIG. 4. However, it is not necessary that the D and E rings be intact during these oxidation, formylation and reduction reactions. For example, in one aspect the present invention provides a method comprising exposing a compound of formula III to silylating conditions to provide a compound of formula V 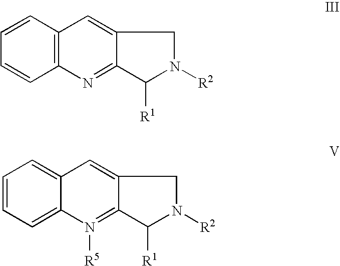
wherein R¹ and R² are the same or different and are independently hydrogen, hydroxyl or an organic group, and R⁵ represents a silyl group, wherein the compound of formula V is associated with a counterion. The terms “organic group” and “counterion” are as previously defined. In one embodiment, R¹ and R² together with the atoms to which they are attached form a heterocycle. In another embodiment, R¹ and R² together with the atoms to which they are attached form the D and E rings of camptothecin, in which case, formula V is compound 14.

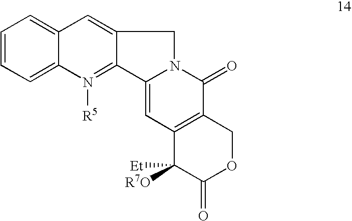

In another aspect, the present invention provides a method comprising exposing a compound of formula V to oxidation conditions. The oxidation step introduces a hydroxyl group to the C10 position while simultaneously de-silylates the N1 position to afford a compound of formula II 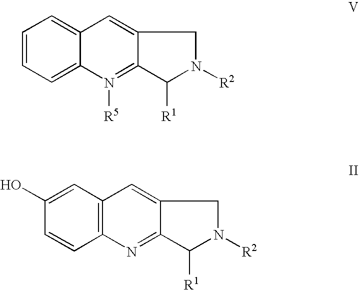
wherein R¹ and R² are the same or different and are independently hydrogen, hydroxyl or an organic group, and R⁵ represents at silyl group, where the compound of formula V is associated with a counterion. The oxidizing reagent can be palladium diacetate, lead(IV) acetate, Jones reagent, or pyridinium chlorochromate.

In a preferred embodiment, compound 14 is exposed to oxidation conditions as set forth above to afford compound 9.

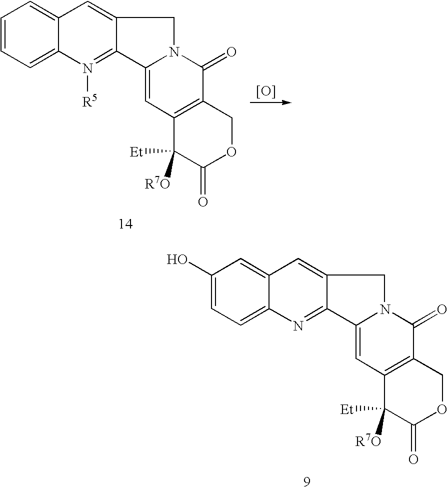

In another aspect, the present invention provides a method comprising exposing a compound of formula II to formylation conditions, to afford a compound of formula VII: 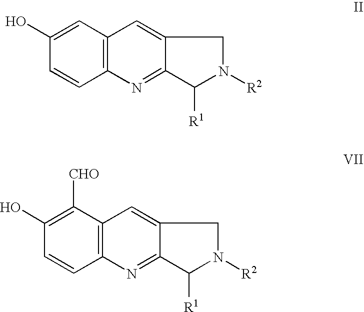
wherein R¹ and R² are the same or different and are independently hydrogen, hydroxyl or an organic group. In one embodiment, R¹ and R² together with the atoms to which they are attached form a heterocycle. In another embodiment, R¹ and R² together with the atoms to which they are attached form the D and E rings of camptothecin. In this incidence, formula II and VII are compounds 9 and 16, respectively.

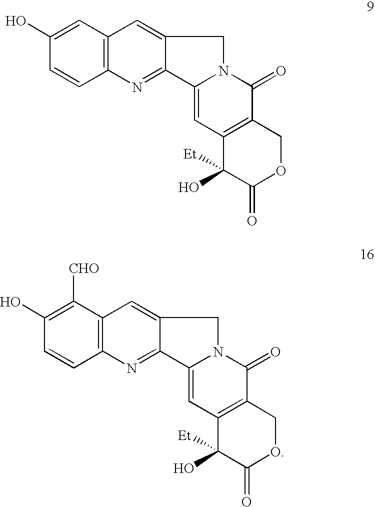

An exemplary formylation condition comprises treating compound of formula II with formaldehyde in the presence of a primary or secondary amine.

In another aspect, the present invention provides a method comprising exposing a compound of formula VII to reductive amination conditions, to afford a compound of formula VIII 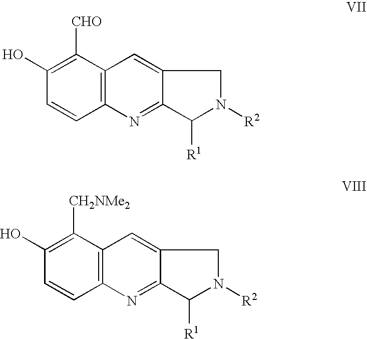
wherein R¹ and R² are the same or different and are independently hydrogen, hydroxyl or an organic group. In one embodiment, R¹ and R² together with the atoms to which they are attached form a heterocycle. In another embodiment, R¹ and R² together with the atoms to which they are attached form the D and E rings of camptothecin, in which case, formula VIII is topotecan 3. 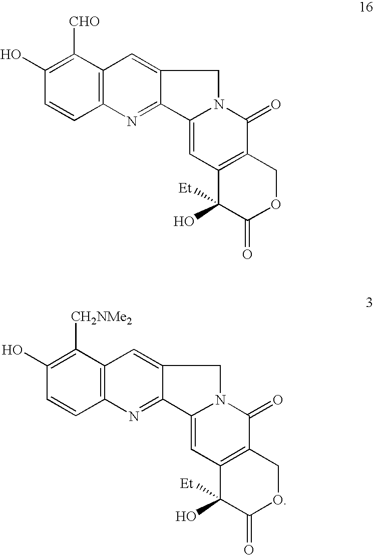
N1-Oxide Camptothecin Derivatives and Related Reactions

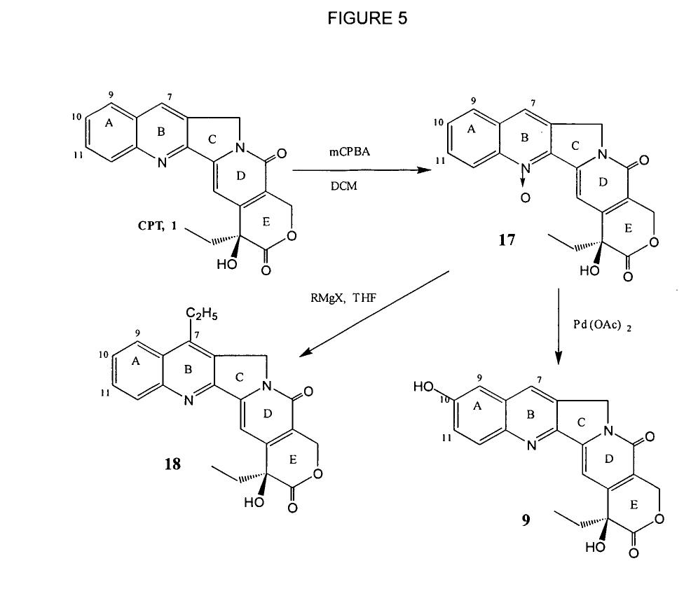

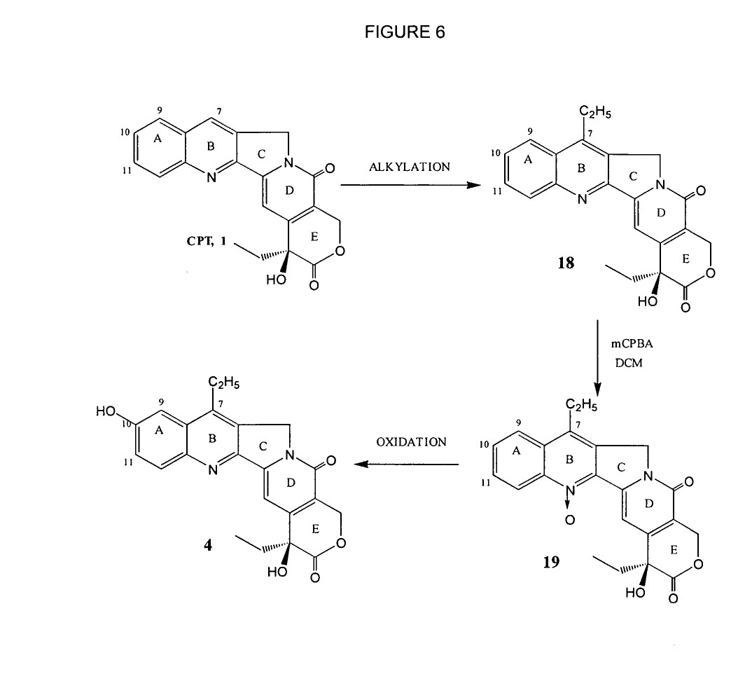

In another aspect, the present invention provides for the oxidation of a starting material selected from camptothecin, its derivatives thereof to form the corresponding 1-oxide compound. The oxidation reaction is generally described by the following scheme, wherein R⁴ is hydrogen or an alkyl group, and [O] represents the 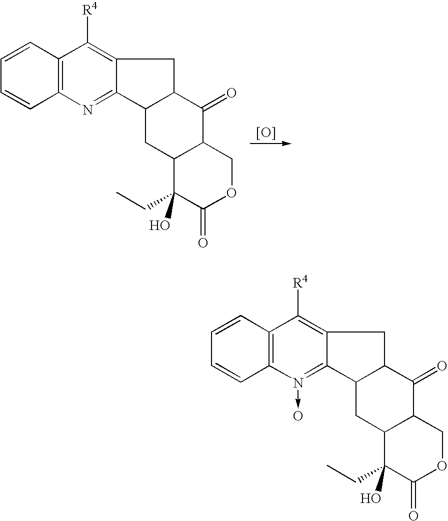
oxidation reactant(s). The oxidation reactant(s) according to the present invention is an organic peroxide or a peroxy acid. In one embodiment, R⁴ is hydrogen. In another embodiment, R⁴ is a C₁-C₆ alkyl. In yet another embodiment, R⁴ is ethyl. A preferred organic peroxide of the present invention is meta-chloroperbenzoic acid (MCPBA). The oxidation reaction is typically conducted in a solvent, where a preferred solvent is dichloromethane (DCM). Exemplary oxidation reactions of the present invention are illustrated in FIG. 5, with the conversion of camptothecin 1 to the corresponding 1-oxide compound 17, and in FIG. 6, with the conversion of compound 18 to compound 19.

In another aspect, the present invention provides for the oxidation of camptothecin-1-oxide or a derivative thereof in which a hydroxyl group is introduced to the C10 position while the oxide group is removed. The oxidation reaction is generally described by the following scheme, wherein R⁴ is hydrogen or an alkyl group, and [O] represents the oxidation 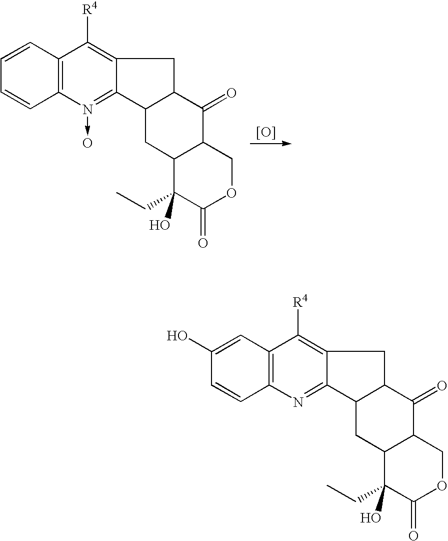
condition(s). According to the present invention, the oxidation conditions(s) employs an oxidizing agent under relatively mild condition, as compared to the prior art method wherein irradiation with UV light is used. Exemplary oxidizing agents can be palladium diacetate, lead(IV) acetate, Jones reagent, or pyridinium chlorochromate. In one embodiment, R⁴ is a C₁-C₆ alkyl. In another embodiment, R⁴ is ethyl. A preferred chemical oxidizing agent is palladium diacetate. The oxidation reaction is typically conducted in a solvent, where a suitable solvent is tetrahydrofuran (THF). Exemplary oxidation reactions of the present invention are illustrated in FIG. 5, with the conversion of 17 to 9, and in FIG. 6, with the conversion of compound 19 to SN-38 4.

In another aspect, the present invention provides for the alkylation of camptothecin, or the 1-oxide derivative of camptothecin, or 10-hydroxy camptothecin to form the correspondence 7-alkyl compound. The alkylation reaction is exemplified by the following Scheme, wherein R⁴ is an 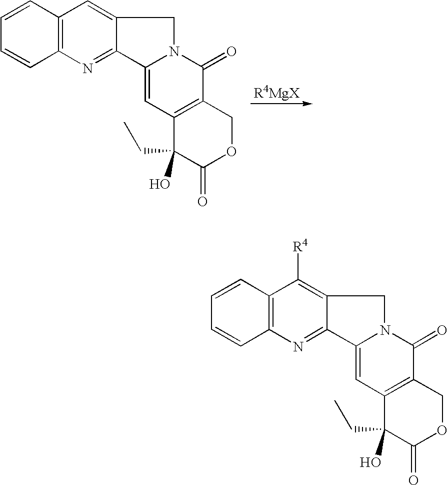
alkyl group, and R⁴MgX represents a Grignard reagent wherein X is a halide. Similarly, camptothecin-1-oxide can be alkylated to afford the corresponding 7-alkyl compound, as illustrated below:

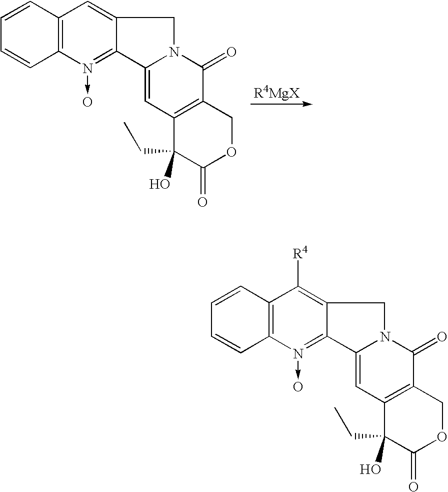

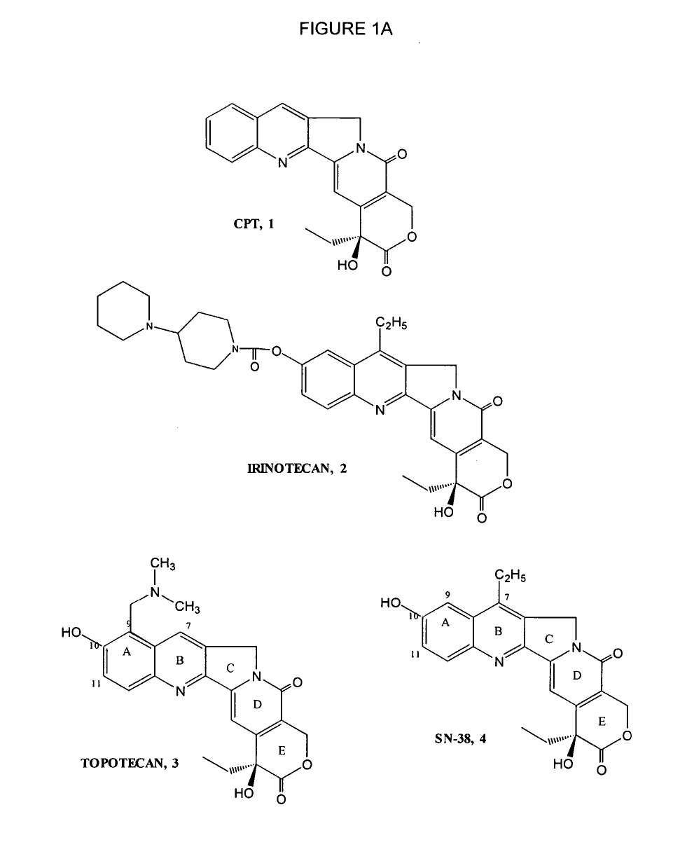

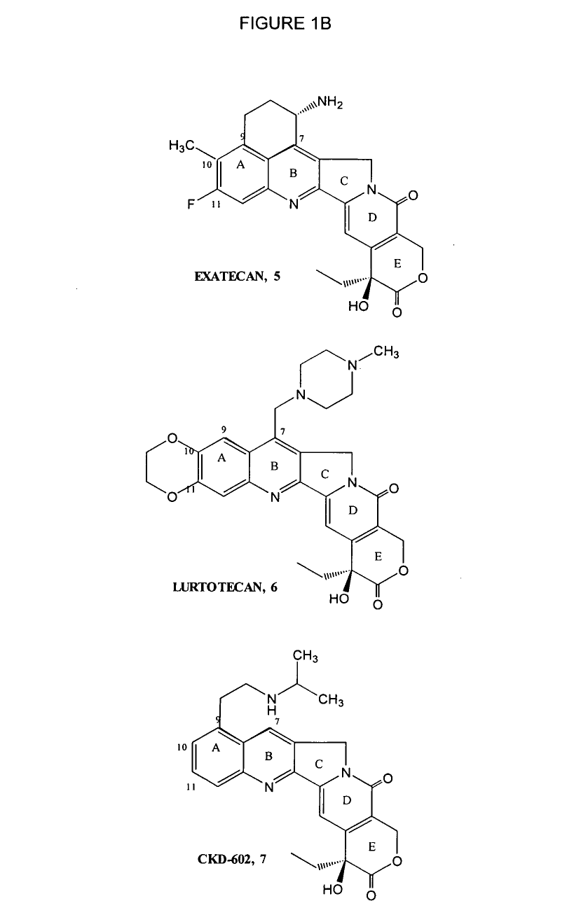

As shown in FIG. 1A, camptothecin 1 has a racemic center at C20 with both ethyl and hydroxyl substitution. The synthetic methods described herein may act on either enantiomer, or on any mixture of enantiomers. For the sake of convenience, some of the structures shown herein do not exhibit any specific stereochemistry. However, the inventive methods apply to all possible enantiomers as well as mixtures of enantiomers, even when the illustrative chemical structures do not indicate specific stereochemistry. For example, in addition to the conversion from 8 to 9 as shown in FIG. 2A where C20 has an S configuration, the inventive method should be considered to more generally provide for the oxidation of compound 8a 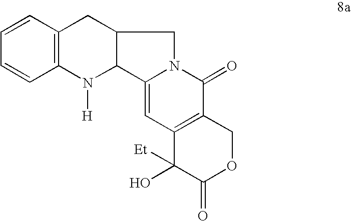
to provide compound 9a, 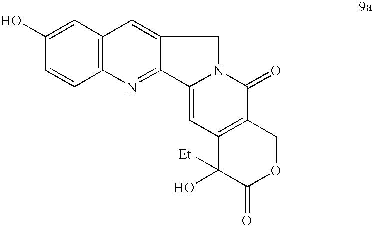
where compounds 8a and 9a denote both racemic and nonracemic mixtures of enantiomers, as well as pure isolated enantiomers. Similarly, the claimed compounds, inspite of the illustration of a particular stereochemistry herein, should be considered to include all possible enantiomers.

The present invention is illustrated by the following non-limiting examples.

The present invention is further illustrated by the following non-limiting examples. Unless otherwise noted, all scientific and technical terms have the meanings as understood by one of ordinary skill in the art.

### EXAMPLES

### Example 1

Camptothecin was hydrogenated using palladium hydroxide in glacial acetic acid at about 50° C. The reaction was monitored by TLC and filtered through celite to get compound 8. The compound 8 was purified by a silica column using mixtures of ethyl acetate/dichloromethane or re-crystallized from ethyl acetate and hexane, and used in the next step. Compound 8 may also be prepared as described in U.S. Pat. No. 4,473,692 (example 13).

### Example 2

To compound 8 in a rapidly stirred suspension of acetic acid at room temperature, was added palladium diacetate or lead (IV) acetate over 10 minutes and the reaction monitored by TLC for complete consumption of starting material. After all the starting material was consumed the reaction was worked up as usual to afford compound 9, that could be further used in the synthesis.

### Example 3

Compound 9 was dissolved in DCM or THF and TBDMSOTf was added and the reaction stirred at room temperature for a couple of hours. The product was used without purification as described in either of Examples 4 or 5.

### Example 4

After the complex from Example 3 was formed as evidenced by TLC, Grignard reagent (ethylmagnesium bromide or ethylmagnesium chloride) in THF was added to the suspension at low temperature and the mixture stirred for an additional 2-3 hours or complete consumption of the starting material at room temperature under argon atmosphere. The reaction was worked up and dissolved in THF and oxygen was bubbled through the solution to afford compound 4 after extraction with hydrochloric acid (1N) and neutralization with aqueous sodium hydroxide.

### Example 5

The mixture prepared in Example 3 was dissolved in THF and the mixture cooled to low temperature at about −40° C. Ethylmagnesium bromide/chloride was added over the course of about 15-30 minutes, keeping the internal reaction temperature at less than −30° C. The cooling bath was removed, and the resulting mixture was allowed to warm to 0° C. and stirred at 0° C. for 1 hour or complete consumption of the starting material and worked up as usual and purified to give compound 4.

### Example 6

The compound 4 was dissolved in pyridine and reacted with 4-piperidinopiperidinecarbamyl chloride dissolved in DCM. The DCM and pyridine are removed by distillation and the crude mixture was worked up by dissolving the crude mixture in DCM and treating with saturated aqueous sodium bicarbonate solution, collecting the organic phase and purifying using column chromatography to afford pure compound 2.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

Finally, it is clear that numerous variations and modifications may be made to process and material described and illustrated herein, all falling within the scope of the invention as defined in the attached claims.

1. A method comprising exposing a compound of formula I to oxidative conditions to provide a compound of formula II 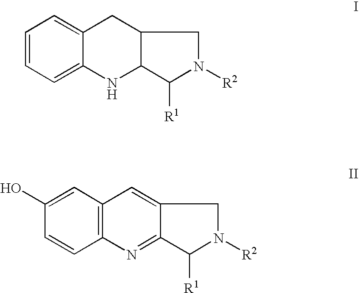
where R¹ and R² are the same or different and are independently hydrogen, hydroxyl and an organic group.

2. The method of claim 1 wherein the organic group is alkyl, alkenyl, alkynyl, alkoxy, acyl, formyl, aryl, heteroaryl or heterocycle.

3. The method of claim 2 wherein R¹ and R² together with the atoms to which they are attached form a heterocycle.

4. The method of claim 3 wherein the heterocycle is a substituted heterocycle.

5. The method of claim 4 wherein the compound of formula I has the structure

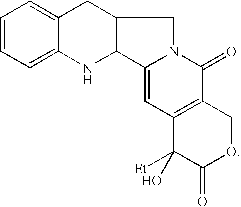

6. The method of claim 4 wherein the compound of formula II has the structure

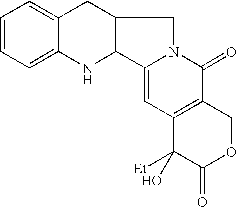

7. The method of claim 1 wherein the oxidation conditions comprise an oxidizing reagent selected from the group consisting of palladium diacetate, lead (IV) acetate, pyridinium chlorochromate (PCC) and Jones reagent.

8. The method of claim 1 wherein 10-hydroxy camptothecin 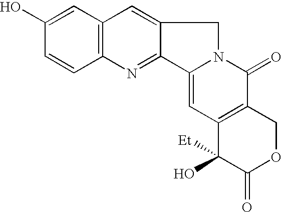
is prepared by treating 1,2,6,7-tetrahydrocamptothecin 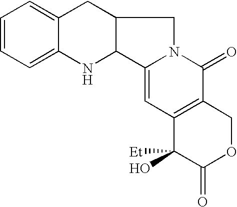
with palladium diacetate or lead (IV) acetate in the presence of a protic acid.

9. The method of claim 8 wherein the protic acid is acetic acid or trifluoroacetic acid.

10. The method of claim 1 in the preparation of irinotecan further comprising converting the compound of formula II into irinotecan.

11. The method of claim 1 in the preparation of topotecan further comprising converting the compound of formula II to topotecan.

12. A method comprising subjecting a compound of formula IIa to silylation conditions to provide a compound of formula IIIa, 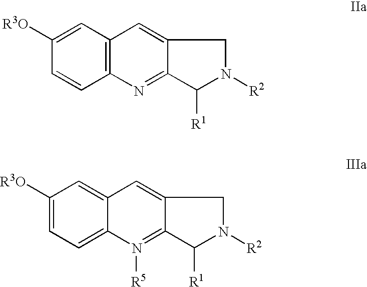
wherein:
R¹ and R² are the same or different and are independently hydrogen, hydroxyl or an organic group;
R³ is hydrogen or a hydroxyl protecting group;
R⁵ is a silyl group;
and wherein the compound of formula IIIa is associated with a counterion.

13. The method of claim 12 wherein R⁵ is t-butyldimethylsilyl, trimethylsilyl, t-butyldiphenylsilyl, or triisopropylsilyl.

14. The method of claim 12 wherein the counterion is triflate or halide.

15. The method of claim 12 wherein the organic group is alkyl, alkenyl, alkynyl, alkoxy, acyl, formyl, aryl, heteroaryl or heterocycle.

16. The method of claim 15 wherein R¹ and R² together with the atoms to which they are attached form a heterocycle.

17. The method of claim 16 wherein the heterocycle is a substituted heterocycle.

18. The method of claim 17 wherein the compound of formula IIa has the structure

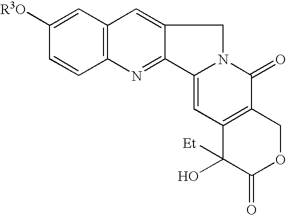

19. The method of claim 17 wherein the compound of formula IIIa has the structure 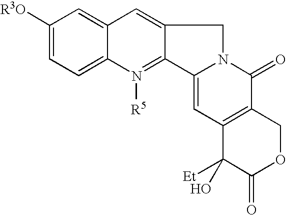
wherein R⁵ is t-butyldimethylsilyl.

20. The method of claim 18 wherein the compound of formula IIa has the structure

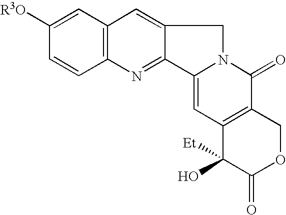

21-106. (canceled)
